(19) United States
(12) Patent Application Publication
(10) Pub. No.: US 2010/0160700 A1
(43) Pub. Date: Jun. 24, 2010

(54) PROCESS AND CATALYSTS FOR REFORMING FISHER TROPSCH NAPHTHAS TO AROMATICS

(75) Inventors: Dennis J O'Rear, Petaluma, CA (US); Cong-Yan Chen, Kensington, CA (US); Stephen J. Miller, San Francisco, CA (US)

Correspondence Address: CHEVRON CORPORATION, P.O. BOX 6006, SAN RAMON, CA, 94583-0806, US

(73) Assignee: Chevron U.S.A. Inc.

(21) Appl. No.: 12/338,436

(22) Filed: Dec. 18, 2008

(51) Int. Cl. C07C 2/00

(52) U.S. Cl. 585/322

(57) ABSTRACT

Improved processes and catalysts are described for the conversion of oxygenate-containing olefinic Fischer Tropsch naphtha into aromatics. This involves removal of the oxygenates without complete saturation of the olefins followed by aromatization of the oxygenate-depleted olefinic naphtha preferably over a catalyst that is tolerant to oxygenates.

### BACKGROUND OF THE INVENTION

Petroleum naphthas can be converted into aromatic products by a two step process that consists of hydrotreating the feedstock to remove sulfur compounds, followed by aromatization over an acidic Group VIII metal (CAS), IUPAC col 10 containing catalyst. The aromatization catalyst is sensitive to sulfur and requires the hydrotreating step. Hydrogen is added to the naphtha during hydrotreating, and hydrogen is produced during the aromatization. The typical catalysts used for aromatization of petroleum C7 and heavier naphthas hydrocarbons consist of a Group VIII metal, typically platinum, on an alumina support with a halogen such as fluorine and chlorine, typically a chloride. The halogen imparts acidity to the catalyst which is needed for aromatization reactions. The chloride can easily be stripped from the catalyst when water or oxygenates are in the feed. Thus typical aromatization catalysts require that the water (or oxygenate) content be less than 10 ppm. Other elements such as rhenium or iridium can be added to increase catalyst stability and yield. Conventional reforming catalysts selectively convert cycloparaffins (naphthenes) into aromatics, but show lower selectivities for conversion of paraffins.

Naphthas from the Fischer Tropsch process are different from petroleum naphthas. Fischer Tropsch naphthas are typically devoid of sulfur i.e. less than 3 ppm, but contain oxygenates and olefins. They also contain few if any cycloparaffins and are composed primarily of linear paraffins and olefins. When the conventional aromatization technology is used to process naphtha from a Fischer Tropsch process, hydrotreating must be used to remove the oxygenates that would otherwise strip the chloride from the reforming catalyst and possibly sinter the Group VIII metal (CAS), Col 10 IAPAC. In addition to removing the oxygenates from the naphtha, the hydrotreating converts the olefins into paraffins, which are the least selective hydrocarbon for conversion into aromatics over conventional aromatization catalysts. Hydrotreating also consumes valuable hydrogen.

It would be desirable to adapt and improve a conventional reforming process for processing Fischer Tropsch naphtha include to include removing the oxygenates without significant saturation of the olefins to paraffins, and converting the oxygenate-depleted olefin-containing naphtha into aromatics.

### DESCRIPTION OF THE INVENTION INCLUDING PREFERRED EMBODIMENTS

We have discovered an upgrading technology that removes at least a portion of oxygenates from Fischer Tropsch naphthas without saturation of all the olefins. Furthermore it does not use expensive hydrogen. We have also discovered that Pt on zeolite catalysts can be used to aromatize the oxygenate-depleted olefinic naphtha.

For avoidance of doubt the following terms will be used throughout the specification and will have the following meanings unless otherwise indicated.

The naphtha means a mixture of hydrocarbons containing at least some compounds with between 6 and 10 carbon atoms.

The term “paraffin” means a saturated straight or branched chain hydrocarbon (i.e., an alkane).

The term “olefins” means an unsaturated straight or branched chain hydrocarbon having at least one double bond (i.e., an alkene).

The term “olefinic naphtha” means a naphtha containing a detectable amount of olefins, preferably 2 to 80 wt % olefins, and 20 to 98 wt % non-olefins. The non-olefins are substantially comprised of paraffins. More preferably, olefinic naphthas contains greater than or equal to 10 wt % olefins, even more preferably greater than 25 wt % olefins and most preferably greater than 50 wt % olefins. Preferably, the olefinic naphthas also contains less than 10 ppm sulfur and less than 10 ppm nitrogen, and more preferably both sulfur and nitrogen are less than 5 ppm and even more preferably less than 1 ppm. Preferably the olefinic naphthas contains less than 10 wt % aromatics, more preferably less than 5 wt % aromatics, and even more preferably less than 2 wt % aromatics. Olefins and aromatics are preferably measured by SCFC (Supercritical Fluid Chromatography).

The term “linear primary olefins” means a straight chain 1-alkene, commonly known as alpha olefins.

The term “total acid number” or “acid value” is a measurement of acidity. It is determined by the number of milligrams of potassium hydroxide required for the neutralization of acids present in 1 gram of the sample being measured (mg KOH/g), as measured by ASTM D 664 or a suitable equivalent. The blended distillate fuel according to the present invention preferably has a total acid number of less than 1.5 mg KOH/g and more preferably less than 0.5 mg KOH/g.

The term “oxygenates” means a hydrocarbon containing oxygen, i.e., an oxygenated hydrocarbon. Oxygenates include alcohols, ethers, carboxylic acids, esters, ketones, and aldehydes, and the like.

The term oxygenate-containing means a material or composition with a detectable level of oxygenates.

The term oxygenate-depleted means a material or composition which a reduced amount of oxygenates when compared to the material from which it was prepared.

The term “derived from a Fischer-Tropsch process” or “Fischer-Tropsch derived” means that the product, fraction, or feed originates from or is produced at some stage by a Fischer-Tropsch process.

A hydrocarbon asset provides the source materials for the formation of synthesis gas for the Fischer Tropsch process.

Synthesis gas which is a mixture of H₂ and CO can be made from any hydrocarbon containing asset, i.e. hydrocarbonaceous asset, such as methane, coal, biomass, tar sands, bitumen, shale oil, municipal and agricultural wastes, petroleum fractions, combinations thereof, etc.

An aspect of the present invention is the oxygenate reduction process where the upgrading or purification process is performed to remove oxygenates, including acids, and dissolved metals, and provides an olefinic naphtha with acceptable oxygenate content. The upgrading process is performed by contacting the oxygenate-containing feedstock with a metal oxide catalyst at elevated temperatures. In contacting the oxygenate-containing feedstock with the metal oxide at elevated temperatures, acids are converted into paraffins and olefins by decarboxylation. In addition, alcohols are converted into additional olefins by dehydration, and other oxygenates (including ethers, esters, and aldehydes found at relatively smaller amounts) are converted into hydrocarbons. In this process for upgrading the oxygenate-containing feedstock, expensive hydrogen is not needed; however, it can be used if desired (to improve catalyst/distillate fuel contacting or for heat control). The oxygen in the oxygenate-containing feedstock is converted into water and carbon dioxide, which can easily be separated from the product oxygenate-depleted olefinic naphtha.

Any dissolved metals, such as aluminum, which are present in the olefinic distillate fuel are simultaneously removed and deposited on the metal oxide catalyst. Typically, the metal oxide catalysts used in the upgrading process according to the present invention will show low deactivation rates; however, eventually the catalysts will need to be regenerated or replaced. Regeneration of the catalysts can be accomplished by stripping with a high temperature gas (hydrogen or other), or by burning the catalyst while it is in contact with an oxygen containing gas at elevated temperatures. Regeneration by burning is preferred.

Preferably the upgrading process according to the present invention is performed by passing the oxygenate-containing feedstock through a purification unit containing a metal oxide under conditions of 450 to 800° F., less than 1000 psig, and 0.25 to 10 LHSV without added gaseous components. Preferably, the metal oxide is selected from the group consisting of alumina, silica, silica-alumina, zeolites, clays, and mixtures thereof. Additional components can be added to the metal oxide to promote the dehydration or to retard olefin isomerization. Another alternative is having any added acidity through silica-alumina supports. These catalysts have a lending to facet, i.e. de-activate, faster. Examples of such additional components are basic elements such as Group I or group II elements of the periodic table. These basic components can also retard catalyst fouling. Usually, these components are incorporated into the oxide in the form in the finished catalyst.

By way of example, the upgrading process may be performed by passing the oxygenate-containing feedstock downflow through a purification unit containing a metal oxide at elevated temperatures.

The severity of the upgrading or purification process can be varied as necessary to achieve the desired oxygenate content. Typically the severity of the process is varied by adjusting the temperature, and LHSV. Accordingly, a more severe purification may be accomplished by running the purification process at a higher temperature, and under these more severe purification conditions more oxygenates will be removed thus providing an oxygenate-depleted olefinic naphtha with a lower oxygenates content. Preferably the upgrading or purification process is conducted at a temperature of 600 to 800° F. Preferably the upgrading or purification process is conducted at a LHSV of 0.5 to 2.

The upgrading processes of the present invention provides an oxygenate-depleted olefinic naphtha with an oxygenate content of less than 1 weight percent, without saturating the olefins contained therein. In addition, the upgrading processes preferably provide an oxygenate-depleted olefinic naphtha with a total acid number preferably less than 1.5 mg KOH/g, more preferably less than 1.0 mg KOH/g, and even more preferably less than 0.5 mg KOH/g, without significantly saturating the olefins contained therein. The upgrading processes of the present invention for example remove more than 75, more preferably more than 80, and even more preferably more than 90 weight percent of the oxygenates in the oxygenate-containing feedstock. Accordingly, the upgrading process according to the present invention comprises conditions of an oxygenate conversion of greater than 75%, more preferably greater than 80%, and even more preferably greater than 90%. The upgrading process of the present invention preferably reduces the acid number of the oxygenate-containing feedstock by at least 25%, more preferably by at least 50% and even more preferably by at least 75%.

When the level of oxygenate is reduced below 50%, preferably less than 20 wt %, most preferably less than 1% conventional reforming technologies can be used.

If the oxygenate content is above this, then a reforming catalyst using an intermediate pore zeolite as described in U.S. Pat. No. 5,358,631, and completely incorporated herein by reference should be employed. For example an intermediate pore zeolite can have an intermediate pore size crystalline silicate having a silica to alumina mole ratio of about 200 or greater; and an alkali content of less than 6000 ppm in the crystalline silicate; and an alkali to aluminum ratio in the crystalline silicate between 1 and 5 on a molar basis, in conjunction with a noble metal.

It should be noted that the oxygenate-containing olefinic feedstock used in this invention can be blended with other streams prior to oxygenate reduction or prior to reforming. These include naphthas from hydrocracking, hydrotreating, and catalytic dewaxing of heavier stream; as well as naphthas from the asset itself (such as condensates). However, if used as a blend, these streams must have sulfur contents below 10 ppm, preferably below 1 ppm, and most preferably below 0.1 ppm.

It should also be noted that the hydrogen produced in this aromatization process can be used for other processing steps including but not limited to hydrocracking, hydrotreating, catalytic dewaxing, reduction in CO₂ to CO or methane, fuel, catalyst regeneration, and combinations.

It should further be noted that both the deoxygenation and reforming processes are endothermic. At least a portion of the heat for these processes could be obtained from other high temperature streams such as exit gases from a syngas generation step of a Fischer-Tropsch process. Heat can also be obtained by combustion of hydrogen from the aromatization process, unreacted gases from the Fischer Tropsch process, or other streams, and the like.

Processes for converting paraffin-rich streams into aromatics are well known in the field. Commonly, such conversion processes referred to as “naphtha reforming processes,” are divided into two classes.

The first class of naphtha reforming processes are referred to as “conventional reforming processes.” Conventional reforming processes use a catalyst composed, for example, of Pt, alumina, and a halogen, typically Cl, and further typically comprising Re or Ir. Generally, the catalyst is sulphided, i.e. exposed to sulfur prior to being used in the reaction. Conventional processes for hydrocarbons expose conventional reforming catalysts to sulfur generally less than 10 ppm sulfur prior to use in the reaction to obtain highly selective conversion of C8-10 paraffins into aromatics. High levels of sulfur (>10 ppm) exposure to the catalyst generate poor selectivity for the conversion of C8-10 paraffins into aromatics. In addition, conventional reforming catalysts are not very selective for the conversion of hexane and heptane to aromatics. The maximum level of oxygenate concentration in a feed should be under 100 ppm.

The second class of naphtha reforming processes is referred to as “non-acidic zeolitic reforming processes” such as, for example, the AROMAX® reforming process. Non-acidic zeolitic reforming processes use a catalyst comprising Pt, a non-acidic zeolite, typically an L-zeolite, K, optionally Ba, mixtures thereof and the like. Generally, non-acidic zeolitic reforming catalysts are not exposed to sulfur prior to operation. In addition, non-acidic zeolitic reforming catalysts are highly selective for the conversion of hexane and heptane into aromatics.

The present invention can employ either or both of the above naphtha reforming processes. Aromatic products produced by the above reforming processes can be used in various applications including, but not limited to, high octane blend components for gasolines, typically including a mixture of C₆-C₁₀ aromatics, benzene for use in chemicals, especially for use in the production of cyclohexane, ethylbenzene and/or cumene, toluene for use as a chemical and xylenes for use as chemicals, especially for the production of paraxylene.

As described, the present invention uses an intermediate pore size crystalline silicate material having a high silica to alumina ratio. For example an intermediate pore size crystalline silicate having a silica to alumina mole ratio of about 200 or greater; and an alkali content of less than 6000 ppm in the crystalline silicate; and an alkali to aluminum ratio in the crystalline silicate between 1 and 5 on a molar basis as discussed in U.S. Pat. No. 5,358,631. One preferred material is silicalite or high molar ratio silica to alumina form of ZSM-5. The X-ray diffraction pattern for ZSM-5 is found in Table 1 of U.S. Pat. No. 3,702,886, the specification of which is completely incorporated herein by reference.

Also as reported in the Argauer patent (U.S. Pat. No. 3,702,886), the values in Table 1 were determined by standard techniques. The radiation was the K-alpha doublet of copper, and a scintillation counter spectrometer with a strip chart pen recorder was used. The peak heights, I, and the positions as a function of 2 times theta, where theta is the Bragg angle, were read from the spectrometer chart. From these, the relative intensities, 100 I/Iₒ, where Iₒ is the intensity of the strongest line or peak, and d (obs.), the interplanar spacing in A, corresponding to the recorded lines, were calculated. In Table 1, the relative intensities are given in terms of the symbols s.=strong, m.=medium, m.s.=medium strong, m.w.=medium weak and v.s.=very strong. It should be understood that this X-ray diffraction pattern is characteristic of all the species of ZSM-5 compositions. Ion exchange of the sodium ion with cations reveals substantially the same pattern with some minor shifts in interplanar spacing and variation in relative intensity. Other minor variations can occur depending on the silicon to aluminum ratio of the particular sample, as well as if it had been subjected to thermal treatment.

ZSM-5 is regarded by many to embrace “silicalite” as disclosed in U.S. Pat. No. 4,061,724 to Grose et al, the specification of which is completely incorporated herein by reference. For ease of reference herein, silicalite is referred to as a ZSM-5-type material with a high silica to alumina molar ratio and is regarded as embraced within the ZSM-5 X-ray diffraction pattern. The silica to alumina ratio is on a molar basis of silica (SiO₂) to alumina (Al₂O₃).

Various references disclosing silicalite and ZSM-5 are provided in U.S. Pat. No. 4,401,555 to Miller. These references include the aforesaid U.S. Pat. No. 4,061,724 to Grose et al.; U.S. Pat. No. 29,948 to Dwyer et al.; Flanigan et al., Nature, 271,512-516 (Feb. 9, 1978) which discusses the physical and adsorption characteristics of silicalite; and Anderson et al., J. Catalysis 58,114-130 (1979) which discloses catalytic reactions and sorption measurements carried out on ZSM-5 and silicalite. The disclosures of these references and U.S. Pat. No. 4,401,555 are completely incorporated herein by reference for all purposes, and particularly including their disclosures on methods of making high silica to alumina crystalline silicates having an X-ray diffraction pattern in substantial accord with Table 1 of U.S. Pat. No. 3,702,886.

Other crystalline silicates which can be used in the process of the present invention include those as listed in U.S. Pat. No. 4,835,336; namely: ZSM-11, ZSM-22, ZSM-23, ZSM-35, ZSM-38, ZSM-48, and other similar materials. ZSM-5 is more particularly described in U.S. Pat. No. 3,702,886 and US Re. 29,948, the entire contents of which are incorporated herein by reference for all purposes. ZSM-11 is more particularly described in U.S. Pat. No. 3,709,979 the entire contents of which are incorporated herein by reference for all purposes. Bibby et al., Nature, 280, 664-665 (Aug. 23, 1979) reports the preparation of a crystalline silicate called “silicalite-2”.

ZSM-22 is more particularly described in U.S. Pat. Nos. 4,481,177, 4,556,477 and European Patent 102,716, the entire contents of each being expressly incorporated herein by reference for all purposes. ZSM-23 is more particularly described in U.S. Pat. No. 4,076,842, the entire contents of which are incorporated herein by reference for all purposes. ZSM-35 is more particularly described in U.S. Pat. No. 4,016,245, the entire contents of which are incorporated herein by reference for all purposes. ZSM-38 is more particularly described in U.S. Pat. No. 4,046,859, the entire contents of which are incorporated herein by reference for all purposes. ZSM-48 is more particularly described in U.S. Pat. No. 4,397,827 the entire contents of which are incorporated herein by reference for all purposes. Of these, ZSM-5, ZSM-11, ZSM-22 and ZSM-23 are preferred. ZSM-5 is most preferred for use in the catalyst of the present invention.

Additionally, zeolites SSZ-20 and SSZ-23 are preferred. SSZ-20 is disclosed in U.S. Pat. No. 4,483,835, and SSZ-23 is disclosed in U.S. Pat. No. 4,859,442, both of which are incorporated herein by reference for all purposes.

The crystalline silicate may be in the form of a borosilicate, where boron replaces at least a portion of the aluminum of the more typical aluminosilicate form of the silicate. Borosilicates are described in U.S. Pat. Nos. 4,268,420; 4,269,813; 4,327,236 to Klotz, the disclosures of which patents are incorporated herein for all purposes, and particularly that disclosure related to borosilicate preparation.

In the borosilicate used in the process and catalyst of the present invention, the preferred crystalline structure is that of ZSM-5, in terms of X-ray diffraction pattern. Boron in the ZSM-5 type borosilicates takes the place of aluminum that is present in the more typical ZSM-5 crystalline aluminosilicate structures. Borosilicates contain boron in place of aluminum, but generally there is some trace amounts of aluminum present in crystalline borosilicates.

Still further crystalline silicates which can be used in the present invention are ferrosilicates, as disclosed for example in U.S. Pat. No. 4,238,318, gallosilicates, as disclosed for example in U.S. Pat. No. 4,636,483, and chromosilicates, as disclosed for example in U.S. Pat. No. 4,299,808.

Thus various high silica content silicates (silicates having a high molar ratio of silica to other constituents) can be used as the crystalline silicate component of the catalyst of the present invention.

Borosilicates and aluminosilicates are preferred silicates for use in the present invention. Aluminosilicates are the most preferred. Silicalite is a particularly preferred aluminosilicate for use in the catalyst of the present invention.

As synthesized, silicalite (according to U.S. Pat. No. 4,061,724) has a specific gravity at 77.degree. F. of 1.99.±.0.05 g/cc as measured by water displacement. In the calcined form (600° C. in air for one hour), silicalite has a specific gravity of 1.70.±.0.05 g/cc. with respect to the mean refractive index of silicalite crystals, values obtained by measurement of the as synthesized form and the calcined form (600° C. in air for one hour) are 1.48.±.0.01 and 1.39.±.0.01, respectively.

The X-ray powder diffraction pattern of silicalite (600° C. calcination in air for one hour) has six relatively strong lines (i.e., interplanar spacings). They are set forth in Table A of U.S. Pat. No. 4,061,724, incorporated herein by reference.

Table B of U.S. Pat. No. 4,061,724, incorporated herein by reference shows the X-ray powder diffraction pattern of a typical silicalite composition containing 51.9 mols of SiO₂ per mol of tetrapropyl ammonium oxide (TPA)₂O, prepared according to the method of U.S. Pat. No. 4,061,724, and calcined in air at (1112° F.). for one hour.

Silicalite crystals in both the “as synthesized” and calcined forms are generally orthorhombic and have the following unit cell parameters: a=20.05 A, b=19.86 A, c=13.36 A (all values .±.0.1 A). The pore diameter of silicalite is about 6 .ANG. and its pore volume is 0.18 cc/gram as determined by adsorption. Silicalite adsorbs neopentane (6.2 A kinetic diameter) slowly at ambient room temperature. The uniform pore structure imparts size-selective molecular sieve properties to the composition, and the pore size permits separation of p-xylene from o-xylene, m-xylene and ethyl-benzene as well as separations of compounds having quaternary carbon atoms from those having carbon-to-carbon linkages of lower value (e.g., normal and slightly branched paraffins).

The crystalline silicates of US Re. 29,948, completely incorporated herein by reference, (Reissue of U.S. Pat. No. 3,702,886 to Argauer) are disclosed as having a composition, in the anhydrous state, as follows:

0.9±0.2[XR₂O+(1-X)M_{2/n}O]:<0.005 Al₂O₃:>SiO₂

Where M is a metal, other than a metal of Group IIIA, n is the valence of said metal, R is an alkyl ammonium radical, and x is a number greater than 0 but not exceeding 1. The crystalline silicate is characterized by the X-ray diffraction pattern of Table 1, in Re 29,948.

The crystalline silicate polymorph of U.S. Pat. No. 4,073,865 to Flanigen et al. is related to silicalite and, for purposes oft he present invention, is regarded as being in the ZSM-5 class. The crystalline silicate exhibits the X-ray diffraction pattern in Table A of U.S. Pat. No. 4,073,865, incorporated herein by reference for all purposes.

According to the August 1979 Nature reference cited above, a silicalite-2 precursor can be prepared using tetra-n-butylammonium hydroxide only, although adding ammonium hydroxide or hydrazine hydrate as a source of extra hydroxyl ions increases the reaction rate considerably. It is stable at extended reaction times in a hydrothermal system. In an example preparation, 8.5 mol SiO₂ as silicic acid (74% SiO₂) is mixed with 1.0 mol tetra-n-butylammonium hydroxide, 3.0 mol NH.subA OH and 100 mol water in a steel bomb and heated at 338.degree. F. for three days. The precursor crystals formed are ovate in shape, approximately 2-3 microns long and 1-1.5 microns in diameter. It is reported that the silicalite-2 precursor will not form if Li, Na, K, Rb or Cs ions are present, in which case the precursor of the U.S. Pat. No. 4,061,724 silicalite is formed. It is also reported that the size of the tetraalkylammonium ion is critical because replacement of the tetra-n-butylammonium hydroxide by other quaternary ammonium hydroxides (such as tetraethyl, tetrapropyl, triethylpropyl, and triethylbutyl hydroxides) results in amorphous products. The amount of Al present in silicalite-2 depends on the purity of the starting materials and is reported as being less than 5 ppm. The precursor contains occluded tetraalkylammonium salts which, because of their size, are removed only by thermal decomposition. Thermal analysis and mass spectrometry show that the tetraalkylammonium ion decomposes at approximately 572° F. and is lost as the tertiary amine, alkene and water. This is in contrast to the normal thermal decomposition at 392° F. of the same tetraalkylammonium salt in air.

The Nature article further reports that the major differences between the patterns of silicalite and silicalite-2 are that peaks at 9.06, 13.9, 15.5, 16.5, 20.8, 21.7, 22.1, 24.4, 26.6 and 27.0° theta. (CuK alpha radiation) in the silicalite X-ray diffraction pattern are absent from the silicalite-2 pattern. Also, peaks at 8.8, 14.8, 17.6, 23.1, 23.9 and 29.9 degrees are singlets in the silicalite-2 pattern rather than doublets as in the silicalite pattern. These differences are reported as being the same as those found between the aluminosilicate diffraction patterns of orthorhombic ZSM-5 and tetragonal ZSM-11. Unit cell dimensions reported as calculated on the assumption of tetragonal symmetry for silicalite-2 are a=20.04; b=20.04; c=13.38. The measured densities and refractive indices of silicalite-2 and its precursor are reported as 1.82 and 1.98 g/cc and 1.41 and 1.48 respectively.

For purposes of the present invention, silicalite is regarded as being in the ZSM-5 class, alternatively put, as being a form of ZSM-5 having high silica to alumina molar ratio; silicalite-2 is regarded as being in the ZSM-11 class.

Suitable types of silicalites and their use in reforming hydrocarbons over silicalites are disclosed in U.S. Pat. Nos. 5,558,851; 5,514,362; 5,741,751, 5,182,012, 5,087,347; 5,139,647; and 6,709,644. The complete specifications of the preceding patents are completely incorporated herein by reference for all purposes. In particular the preferred silicalite catalysts and reforming process conditions are incorporated herein by reference.

The preparation of crystalline silicates of the present invention generally involves the hydrothermal crystallization of a reaction mixture comprising water, a source of silica, and an organic templating compound at a PH of 10 to 14. Representative templating moieties include quaternary cations such as XR₄ where X is phosphorous or nitrogen and R is an alkyl radical containing from 2 to 6 carbon atoms, e.g., tetrapropylammonium hydroxide (TPA-OH) or halide, as well as alkyl hydroxyalkyl compounds, organic amines and diamines, and heterocycles such as pyrrolidine.

When the organic templating compound (i.e., TPA-OH) is provided to the system in the hydroxide form insufficient quantity to establish a basicity equivalent to the PH of 10 to 14, the reaction mixture may contain only water and a reactive form of silica as additional ingredients. In those cases in which the pH must be increased to above 10, ammonium hydroxide or alkali metal hydroxides can be suitably employed for that purpose, particularly the hydroxides of lithium, sodium and potassium. The ratio: R⁺ to the quantity R⁺M⁺, where R⁺ is the concentration of organic templating cation and M⁺ is the concentration of alkali metal cation, is preferably between 0.7 and 0.98, more preferably between 0.8 and 0.98, most preferably between 0.85 and 0.98.

The source of silica in the reaction mixture can be wholly, or in part, alkali metal silicate. Other silica sources include solid reactive amorphous silica, e.g., fumed silica, silica sols, silica gel, and organic orthosilicates. One commercial silica source is Ludox AS-30, available from Du Pont.

Aluminum, usually in the form of alumina, is easily incorporated as an impurity into the crystalline silicate. Aluminum in the crystalline silicate contributes acidity to the catalyst, which is undesirable. To minimize the amount of aluminum, care should be exercised in selecting a silica source with a minimum aluminum content.

Commercially available silica sols can typically contain between 500 and 700 ppm alumina, whereas fume silicas can contain between 80 and 2000 ppm of alumina impurity. As explained above, the silica to alumina molar ratio in the crystalline silicate of the catalyst used in the present invention is preferably greater than 500:1, more preferably greater than 1000:1, most preferably greater than 2000:1.

The quantity of silica in the reaction system is preferably between about 1 and 10 mols SiO₂ per mol-ion of the organic templating compound. Water should be generally present in an amount between 20 and 700 mol per mol-ion of the quaternary cation. The reaction preferably occurs in an aluminum-free reaction vessel which is resistant to alkali or base attack, e.g., Teflon.

In forming the final catalyst used in the present invention, the crystalline silicate is may be bound with a matrix. The term “matrix” includes inorganic compositions with which the silicate can be combined, dispersed, or otherwise intimately admixed. Preferably, the matrix is not catalytically active in a hydrocarbon cracking sense, i.e., contains substantially no acid sites. Satisfactory matrices include inorganic oxides. Preferred inorganic oxides include alumina, silica, naturally occurring and conventionally processed clays, for example bentonite, kaolin, sepiolite, attapulgite and halloysite. Preferred matrices are substantially non-acidic and have little or no cracking activity. Silica matrices and also alumina matrices are especially preferred. We have found that the use of a low acidity matrix, more preferably a substantially non-acidic matrix, is advantageous in the catalyst of the present invention.

Compositing the crystalline silicate with an inorganic oxide matrix can be achieved by any suitable method wherein the silicate is intimately admixed with the oxide while the latter is in a hydrous state (for example, as a hydrous salt, hydrogel, wet gelatinous precipitate, or in a dried state, or combinations thereof). One method of manufacture is to prepare a hydrous mono or plural oxide gel or cogel using an aqueous solution of a salt or mixture of salts (for example, aluminum sulfate and sodium silicate). Ammonium hydroxide carbonate (or a similar base) is added to the solution in an amount sufficient to precipitate the oxides in hydrous form. Then, the precipitate is washed to remove most of any water soluble salts and it is thoroughly admixed with the silicate which is in a finely divided state. Water or a lubricating agent can be added in an amount sufficient to facilitate shaping of the mix (as by extrusion).

A preferred crystalline silicate for use in the catalyst of the present invention is ZSM-5 having a high silica to alumina molar ratio, which, for convenience, is frequently referred to herein as “silicalite”. Assuming that the only crystalline phase in the silicalite prep is silicalite, the silicalite preferably has a percent crystallinity of at least 80%, more preferably at least 90%, most preferably at least 95%. To determine percent crystallinity, an x-ray diffraction (XRD) pattern of the silicalite is made and the area under the eight major peaks is measured in the angle interval between 20.5 and 25.0 degrees. Once the area under the curve is calculated, it is compared with the area under the curve for a 100% crystalline standard for silicalite.

The preferred crystallite size of the crystalline silicate is less than 10 microns, more preferably less than 5 microns, still more preferably less than 2 microns, and most preferably less than 1 micron. When a crystallite size is specified, preferably at least 70 wt. % of the crystallites are that size, more preferably at least 80 wt. %, more preferably 90 wt. %. Crystallite size can be controlled by adjusting synthesis conditions, as known to the art. These conditions include temperature, pH, and the mole ratios H₂O/SiO₂, R⁺/SiO₂, and M⁺/SiO₂, where R⁺ is the organic templating cation and M⁺ an alkali metal cation. For small crystallite size, i.e., less than 10 microns, typical synthesis conditions are listed below:

| | | More | Most |
|---|---|---|---|
| | Preferred | Preferred | Preferred |
| Temp (° F.) | 176-392 | 144-356 | 212-302 |
| pH | 12-14 | 12.5-14 | 13-13.5 |
| H₂O/SiO₂ | 5-100 | 10-50 | 10-40 |
| R⁺/SiO₂ | 0.1-1.0 | 0.1-0.5 | 0.2-0.5 |
| M⁺/SiO₂ | 0.01-0.3 | 0.01-0.15 | 0.01-0.08 |

Other techniques known to the art, such as seeding with silicate crystals, can be used to reduce crystallite size.

The crystalline silicate component of the catalyst of the present invention has an intermediate pore size. By “intermediate pore size” as used herein is meant an effective pore aperture in the range of about 5 to 6.5 Anstroms when the silicate is in the H-form. Crystalline silicates having pore apertures in this range tend to have unique molecular sieving characteristics. Unlike small pore crystalline silicates or zeolites such as erionite, they will allow hydrocarbons having some branching into the zeolitic void spaces. Unlike large pore zeolites such as the faujasites, they can differentiate between n-alkanes and slightly branched alkanes on the one hand and larger branched alkanes having, for example, quarternary carbon atoms.

The effective pore size of the crystalline silicates or zeolites can be measured using standard adsorption techniques and hydrocarbonaceous compounds of known minimum kinetic diameters. See Breck, Zeolite Molecular Sieves, 1974 (especially Chapter 8) and Anderson et al., J. Catalysis 58,114 (1979), both of which are incorporated by reference.

Intermediate pore size crystalline silicates or zeolites in the H-form will typically admit molecules having kinetic diameters of 5 to 6 Anstroms with little hindrance. Examples of such compounds (and their kinetic diameters in Angstroms) are: n-hexane (4.3), 3-methylpentane (5.5), benzene (5.85), and toluene (5.8). Compounds having kinetic diameters of about 6 to 6.5 Angstroms can be admitted into the pores, depending on the particular zeolite, but do not penetrate as quickly and in some cases, are effectively excluded (for example, 2,2-dimethylbutane is excluded from H-ZSM-5). Compounds having kinetic diameters in the range of 6 to 6.5 Angstroms include: cyclohexane (6.0), m-xylene (6.1) and 1,2,3,4-tetramethylbenzene (6.4). Generally, compounds having kinetic diameters of greater than about 6.5 Angstroms cannot penetrate the pore apertures and thus cannot be adsorbed in the interior of the zeolite. Examples of such larger compounds include: o-xylene (6.8), hexamethylbenzene (7.1), 1,3,5-trimethylbenzene (7.5), and tributylamine (8.1).

The preferred effective pore size range is from about 5.3 to about 6.2 Angstroms. ZSM-5, ZSM-11 and silicalite, for example, fall within this range.

In performing adsorption measurements to determine pore size, standard techniques are used.

Examples of intermediate pore size zeolites include silicalite and members of the ZSM series such as ZSM-5, ZSM-11, ZSM-21, ZSM-22, ZSM-23, ZSM-35, ZSM-38, SSZ-20 and SSZ-23.

The catalysts according to the present invention contain one or more noble metals. Preferred metals are rhodium, palladium, iridium or platinum. Palladium, and platinum are more preferred. Platinum is most preferred. The preferred percentage of the noble metal, such as platinum, in the catalyst is between 0.1 wt. % and 5 wt. %, more preferably from 0.3 wt. % to 2.5 wt. %.

Noble metals are preferably introduced into the crystalline silicate by impregnation, occlusion, or ion exchange in an aqueous of an appropriate salt. When it is desired to introduce two Group VIII metals into the crystalline silicate, the operation may be carried out simultaneously or sequentially. Preferably, the Group VIII metal is finely dispersed within, and on, the crystalline silicate.

By way of example, platinum can be introduced by impregnation with an aqueous solution of tetraammineplatinum (II) nitrate, tetraammineplatinum (II) hydroxide, dinitrodiamino-platinum or tetraammineplatinum (II) chloride. In an ion exchange process, platinum can be introduced by using cationic platinum complexes such as tetraammineplatinum (II) nitrate or chloride. When platinum is introduced into the silicalite by occlusion, a platinum complex is preferably introduced into the crystalline silicate during its formation.

After platinum impregnation, the catalyst is preferably ammonium exchanged, if necessary, to remove alkali metals.

After the desired metal or metals have been introduced, the catalyst is preferably treated in air, or air diluted with an inert gas, and reduced in hydrogen. Catalysts containing platinum are typically subjected to halogen or halide treatments to achieve or maintain a uniform metal dispersion. Typically, the halide is a chloride compound. The catalysts of our invention can be subjected to similar treatments although the preferred catalyst does not contain chloride in the final form.

The catalyst can be employed in any of the conventional types of catalytic reforming or dehydrocyclization equipment. The catalyst can be employed in the form of pills, pellets, granules, broken fragments, or various special shapes within a reaction zone.

The feed to the reformer or dehydrocyclization zone is preferably a light hydrocarbon or naphtha fraction, preferably boiling within the range of about 70° to 600° F. and more preferably from 120° to 400° F. This can include, for example, straight run naphthas, paraffinic raffinates from aromatic extraction, and C₆ and —C₁₀ paraffin-rich feeds, as well as paraffin-containing naphtha products from other refinery processes, such as hydrocracking or conventional reforming. The actual reforming conditions will depend in large measure on the feed used, whether highly aromatic, paraffinic or naphthenic and upon the desired octane rating of the product. The catalyst of the present invention is preferably used to dehydrocyclize acyclic hydrocarbons to form aromatics.

We have found that the catalyst of the present invention has greater stability (for yield and octane maintenance) if the amount of water introduced to the reaction zone is less than 50 ppm by weight, more preferably less than 25 ppm.

In the process of the present invention, the pressure is preferably between 0 psig and 200 psig, more preferably between 0 psig and 100 psig, and most preferably between 25 psig and 75 psig. The liquid hourly space velocity (LHSV) is preferably between about 0.1 to about 20 hr⁻¹ with a value in the range of about 0.3 to about 5 hr⁻¹ being preferred. The temperature is preferably between about 800° F. and about 1100° F., more preferably between 840° F. and 1050° F. As is well known to those skilled in the dehydrocyclization art, the initial selection of the temperature within this broad range is made primarily as a function of the desired conversion level of the acyclic hydrocarbon considering the characteristics of the feed and of the catalyst. Thereafter, to provide a relatively constant value for conversion, the temperature is slowly increased during the run to compensate for the inevitable deactivation that occurs.

In accordance with one embodiment of the present invention, it is not necessary to contact the low alkali catalyst with recycle hydrogen. In this embodiment, the absence of added or recycle hydrogen favors aromatics production and relative activity which increases liquid yield at a given octane.

In accordance with another embodiment of the present invention, some hydrogen is recycled. This increases catalyst life and conserves heat. The preferred recycle hydrogen to fresh feed hydrocarbon mole ratio is generally between 0 and 10, more preferably 0 to 5, most preferably 0.5 to 2. In accordance with the embodiment wherein hydrogen is recycled, the preferred ranges are as specified except with a lower limit of 0.1 recycle hydrogen to fresh feed hydrocarbon mole ratio.

We have found that the low alkali catalysts of the present invention achieve particularly good selectivity to C₅+liquids in reforming or dehydrocyclization if they are presulfided prior to use in reforming or dehydrocyclization. The sulfiding of the catalyst can be carried out in situ (in the reforming or dehydrocyclization reactor or reactors) or ex situ. Preferably, the sulfiding is carried out in situ. Sulfiding techniques known in the art are suitable.

In the reforming process embodiment of the present invention, the hydrocarbon feed is contacted with the catalyst as described above in a reforming zone or reforming reactor under reforming conditions. This contacting can be accomplished by using the catalyst in a fixed-bed system, a moving bed system, a fluidized system or in a batch-type operation; however, it is preferred to use either a fixed-bed system or a dense phase moving bed system.

### EXAMPLES OF EMBODIMENTS OF THE INVENTION

The invention will be further explained by the following illustrative examples that are intended to be non-limiting.

### Example 1

### Fischer-Tropsch Oxygenate-Containing and Olefinic Feedstocks

Two oxygenate-containing olefinic feedstocks prepared by the Fischer-Tropsch process were obtained. The first (Feedstock A) was prepared by use of a iron catalyst. The second (Feedstock B) was prepared by use of a cobalt catalyst. The Fischer-Tropsch process used to prepare both feeds was operated in the slurry phase. Properties of the two feeds are shown below in Table 4 below. These feedstocks contain both a typical naphtha (C₆-C₁₀ hydrocarbons) and distillate (C₁₀+hydrocarbons). For purposes of this application the oxygenates are reduced in this broadly boiling feedstock. They could also be reduced from a lighter oxygenate-containing olefinic Fischer-Tropsch naphtha.

Feedstock A contains significant amounts of dissolved iron and is also acidic. It has a significantly poorer corrosion rating.

For purposes of this invention, Feedstock B is preferable. It contains fewer oxygenates, has a lower acid content, and is less corrosive. Thus it is preferable to prepare oxygenate-depleted and olefinic naphthas for use in blended fuels from cobalt catalysts rather than iron catalysts.

A modified version of ASTM D6550 (Standard Test Method for the Determination of the Olefin Content of Gasolines by Supercritical Fluid Chromatography—SFC) was used to determine the group types in the feedstocks and products. The modified method is to quantify the total amount of saturates, aromatics, oxygenates and olefins by making a 3-point calibration standard. Calibration standard solutions were prepared using the following compounds: undecane, toluene, n-octanol and dodecene. External standard method was used for quantification and the detection limit for aromatics and oxygenates is 0.1 % wt and for olefins is 1.0% wt. Please refer to ASTM D6550 for instrument conditions.

A small aliquot of the fuel sample was injected onto a set of two chromatographic columns connected in series and transported using supercritical carbon dioxide as the mobile phase. The first column was packed with high surface area silica particles. The second column contained high surface area silica particles loaded with silver ions.

Two switching valves were used to direct the different classes of components through the chromatographic system to the detector. In a forward-flow mode, saturates (normal and branched alkanes and cyclic alkanes) pass through both columns to the detector, while the olefins are trapped on the silver-loaded column and the aromatics and oxygenates are retained on the silica column. Aromatic compounds and oxygenates were subsequently eluted from the silica column to the detector in a back flush mode. Finally, the olefins were back flushed from the silver-loaded column to the detector.

A flame ionization detector (FID) was used for quantification. Calibration was based on the area of the chromatographic signal of saturates, aromatics, oxygenates and olefins, relative to standard reference materials, which contain a known mass % of total saturates, aromatics, oxygenates and olefins as corrected for density. The total of all analyses was within 3% of 100% and normalized to 100% for convenience.

The weight percent olefins can also be calculated from the bromine number and the average molecular weight by use of the following formula:

Wt % Olefins=(Bromine No.)(Average Molecular Weight)/159.8.

It is preferable to measure the average molecular weight directly by appropriate methods, but it can also be estimated by correlations using the API gravity and mid-boiling point as described in “Prediction of Molecular Weight of Petroleum Fractions” A. G. Goossens, IEC Res. 1996,35, p.985-988. Preferably the olefins and other components are measured by the modified SFC method as described above. A GCMS analysis of the feedstocks determined that the saturates were almost exclusively n-paraffins, and the oxygenates were predominantly primary alcohols, and the olefins were predominantly primary linear olefins (alpha olefins).

### Example 2

### Dehydration Catalysts

Commercial Silica Alumina and Alumina extrudates were evaluated for dehydration of the Olefinic Naphthas. Properties of the extrudates are shown below in Table 1.

TABLE 1

| | Extrudate | |
|---|---|---|
| | Silica Alumina | Alumina |
| Method of manufacture | 89% silica alumina | Alumina |
| | powder bound with | extrudate |
| | 11% alumina | |
| Particle Density, gm/cm3 | 0.959 | 1.0445 |
| Skeletal Density, gm/cm3 | 2.837 | |
| BET Surface area, m2/g | 416 | 217 |
| Geometric Average pore size, | 54 | 101 |
| Angstroms | | |
| Macropore volume, cc/g (1000 + | 0.142 | 0.0032 |
| Angstroms) | | |
| Total pore volume, cc/g | 0.636 | 0.669 |

### Example 3

### Dehydration Over Silica Alumina

The dehydration experiments were performed in one inch downflow reactors without added gas or liquid recycle. The catalyst volume was 120 cc. The Fe-based condensate (Feed A) was treated with the commercial silica-alumina. This catalyst was tested at 50 psig and temperature of 480° F., 580° F., and 680° F. with space velocity at one LHSV and three LHSV. At one LHSV, the total olefin content was 69-70% at all three temperatures, which indicated full conversion of the oxygenates. At 680° F. some cracking was observed by the light product yields: total C4—was 1.2% and C5—290° F. was 25% (vs. 20% in the feedstock). At three LHSV and 480° F. and 580° F. the total olefins were lower at 53-55%. High dehydration activity was obtained at 680° F. and three LHSV with total olefin content of 69%. GCMS data indicated that significant amount of 1-olefin was converted to internal or branched olefins. The total olefins at 480° F. was 69% initially but was 55% near the end of the test (˜960 hours on stream). Significant amount of carbon was observed on the catalyst after unloading the catalyst. The results are in Table 3 and the catalyst apparently fouled to end the test.

TABLE 2

| Dehydration | | | Bromine method | | GC-MS Data |
|---|---|---|---|---|---|
| Si—Al | | | | % | Alpha-olefins/ |
| catalyst | Temp, F. | LHSV | Bromine # | Olefin | Total olefins |
| Sample A | | | 50.6 | 51.6 | 90% |
| Product D | 680 | 3 | 71.7 | 70.3 | 5% |
| | 680 | 1 | 72.2 | 70.5 | 6% |

The detailed analysis of the product (D) from the test at 3 LHSV and 680° F. is shown below in Table 4. 84% of the oxygen was removed, the corrosion rating was improved, and iron was reduced to below the level of detection. The acidity of the naphtha was reduced by 25%. The oxygenates were converted to olefins as shown by the increase in olefin content and the decrease in oxygenate content.

### Example 4

### Dehydration Over Alumina

The Co-based cold condensate (Feedstock B) was also treated as in Example 2, but with the alumina catalyst. Temperatures from 480° F. to 730° F. and LHSV values from one to five were explored. At high temperature and one LHSV, GCMS data indicated that the double bond isomerization was significant (reduced alpha-olefin content). At five LHSV and 580° F., dehydration conversion was significantly lower, and the majority of the olefins were primary linear olefins. This test ran 2000 hours with no indication of fouling. The results are in Table 3.

TABLE 3

| | | | | | | GC-MS | | |
|---|---|---|---|---|---|---|---|---|
| Dehydration | | | SFC | | | Data | | |
| alumina | | | method | | | Alpha- | C4-Gas | Total |
| catalyst | | | Oxygenates, | Bromine method | | olefins/Total | Yields, | Acid |
| Sampe ID | Temp, F. | LHSV | % wt | Bromine# | % Olefin | Olefins | Wt % | No. |
| Feed B | | | 8.5 | 20.4 | 24.2 | 94% | | 0.86 |
| B1 | 480 | 1 | 7.4 | 21.3 | 25.2 | 92% | 0.32 | |
| B2 | 580 | 1 | 0.9 | 27.5 | 31.8 | 85% | <0.5 | |
| B3 | 580 | 1 | 0.8 | 28.2 | 33.1 | 91% | 0.34 | 0.6 |
| B4 | 580 | 1 | 0.9 | 27.1 | 31.1 | 93% | 0.36 | |
| B5 | 580 | 2 | 1.3 | 27.1 | 31.3 | 86% | <0.5 | |
| B6 | 580 | 3 | 2.1 | 26.5 | 30.6 | 86% | <0.5 | 0.48 |
| B7 | 630 | 1 | 0.6 | 27.9 | 32.2 | 78% | 0.46 | 0.32 |
| B8 | 630 | 2 | 0.8 | 28.1 | 32.4 | 79% | 0.38 | |
| B9 | 630 | 3 | 0.8 | 29.4 | 33.9 | 86% | 0.24 | 0.63 |
| B10 | 630 | 4 | 1 | 28.7 | 33.1 | 87% | 0.2 | |
| B11 | 630 | 5 | 1.1 | 27.1 | 31.1 | 83% | 0.18 | 0.67 |
| B12 | 680 | 1 | <0.1 | 31.1 | 35.6 | 4% | 0.51 | 0.06 |
| B13 | 680 | 2 | 0.3 | 26.7 | 30.8 | 30% | 0.4 | 0.18 |
| B14 | 680 | 3 | 0.5 | 26.5 | 30.6 | 71% | 0.33 | |
| B15 | 680 | 3 | 0.6 | 26.9 | 31.1 | 78% | <0.5 | |
| B16 | 680 | 4 | 0.6 | 27.6 | 32.0 | 76% | <0.5 | |
| B17 | 680 | 4 | 0.6 | 29.1 | 33.3 | 73% | 0.2 | |
| Product C | 680 | 5 | 0.7 | 28.1 | 32.3 | 78% | 0.18 | 0.39 |
| C1 | 680 | 5 | 0.7 | 27.8 | 31.9 | 79% | <0.5 | |
| C2 | 730 | 3 | 0.1 | 31.8 | 36.1 | 7% | 0.33 | 0.12 |

These results show that it is possible to eliminate all the oxygenates (measured by SFC) from the sample and convert them to olefins. At high oxygenate removal levels, a significant portion of the alpha olefins are isomerized to internal olefins, but this does not decrease their value as a naphtha for subsequent aromatization.

Product (C) was prepared from operation at five LHSV and 680° F. Detailed properties are shown below in Table 4. 87% of the oxygen is removed, the acidity was reduced by 55%, and the trace of iron in the sample was removed. The acidity of the final material was below 0.5 mg KOH/g, the typical maximum for petroleum crudes. The oxygenates were converted to olefins as shown by the increase in olefin content which approximately matched the decrease in oxygenate content.

A Summary and comparison of the Examples is in Table 4 below:

| | Experiment No. | | | |
|---|---|---|---|---|
| | 1 | 2 | 1 | 3 |
| Feed/Product ID | Fe Condo | Product D | Co Condo B | Product C |
| | A | | | |
| Process conditions | | | | |
| Catalyst | None | SiAl | None | Alumina |
| LHSV, h-1 | — | 3 | — | 5 |
| Temperature, F. | — | 680 | — | 680 |
| Pressure, psig | — | 50 | — | 50 |
| Run hours | — | 582-678 | — | 1026-1122 |
| API | 56.5 | 58.1 | 56.6 | 57.9 |
| Bromine No. | 50.6 | 71.7 | 21 | 27.6 |
| Average molecular | 163 | 157 | 183 | 184 |
| weight | | | | |
| Wt % Olefin (calc. | 51.6 | 70.3 | 24 | 32 |
| from Br2 No.) | | | | |
| KF Water, ppm wt | 494 | 58 | 530 | 57 |
| Oxygen byNAA, wt % | 1.61 | 0.26 | 0.95 | 0.12 |
| SFC Analysis, Wt % | | | | |
| Saturates | 33.5 | 35.1 | 67.4 | 68 |
| Aromatics | 1.2 | 1.5 | 0.3 | 0.4 |
| Olefins | 55.7 | 62.2 | 23.7 | 30.9 |
| Oxygenates | 9.6 | 1.2 | 8.6 | 0.7 |
| Acid Test | | | | |
| Total Acid, mg | 3.17 | 2.33 | 0.86 | 0.39 |
| KOH/g | | | | |
| BUF EP, mg KOH/g | 3.1 | 2.3 | 0.84 | 0.35 |
| Cu Strip Corrosion | | | | |
| Rating | 2c | 2a | 1b | 1b |
| Sulfur, ppm wt | <1 | n/a | <1 | <1 |
| Nitrogen, ppm | 0.56 | n/a | 1.76 | 1.29 |
| ASTM D2887 | 86 | 102 | 76 | 91 |
| Simulated Distillation | | | | |
| by wt %, ° F. | | | | |
| 0.5 | 237 | 214 | 243 | 247 |
| 10 | 301 | 303 | 339 | 338 |
| 30 | 373 | 356 | 415 | 414 |
| 50 | 417 | 417 | 495 | 486 |
| 70 | 484 | 485 | 569 | 572 |
| 90 | 517 | 518 | 596 | 599 |
| 95 | 639 | 622 | 662 | 666 |
| 99.5 | | | | |
| Metals by ICP, ppm | | | | |
| Fe | 44.96 | 0.98 | 2.02 | <0.610 |
| Zn | 2.610 | <0.380 | <0.360 | <0.350 |

Metal elements below ICP limit of detection in all samples:

AI, B, Ba, Ca, Cr, Cu, K, Mg, Mo, Na, Ni, P, Pb, S, Si, Sn, Ti, V.

### Example 5

### Adsorption of Oxygenates

Trace levels of oxygenates not removed by the high temperature treatment can be removed by adsorption using sodium X zeolite (commercial 13X sieve from EM Science, Type 13X, 8-12 Mesh Beads, Part Number MXI583T-1).

The adsorption test was carried out in a up-flow fixed bed unit. The feed for the adsorption studies was produced by processing the Co condensate (Feed B) over alumina at 5 LHSV, 680° F. and 50 psig. The feed for the adsorption studies had acid number of 0.47 and oxygenate content by SFC of 0.6%. Process conditions for the adsorption were: ambient pressure, room temperature, and 0.5 LHSV. The oxygenate content of the treated products was monitored by the SFC method. The adsorption experiment was continued until breakthrough—defined as the appearance of an oxygenate content of 0.1% or higher. The breakthrough occurred at when the sieve had adsorbed an equivalent amount of 14 wt % based on the feed and product oxygenates. The product after treatment showed 0.05 wt % oxygen by neutron activation, <0.1 ppm nitrogen, and total acid number of 0.09. The adsorbent could be regenerated by known methods: oxidative combustion, calcinations in inert atmosphere, water washing, and the like, and in combinations. These results demonstrate that adsorption processes can also be used for oxygenate removal. They can be used as such, or combined with dehydration.

What is claimed is:

1. A process for producing aromatics consisting of:
a) Converting at least a portion of a hydrocarbon asset into synthesis gas;
b) Converting at least a portion of the synthesis gas to an oxygenate-containing hydrocarbon stream in a Fischer Tropsch process unit
c) Treating the oxygenate-containing hydrocarbon stream to obtain an oxygenate-depleted olefinic stream,
d) Aromatizing the oxygenate-depleted olefinic stream, and e) recovering an aromatic product.

2. A process according to claim 1 wherein the aromatization is done by a process selected from the group consisting of conventional reforming, reforming over an intermediate pore zeolite, and combinations.

3. A process according to claim 1 wherein the aromatization is done by a conventional reforming process and wherein the oxygenate-depleted olefinic stream further comprises an oxygen content of less than 10 ppm.

4. The process according to claim 1 wherein the Fischer-Tropsch process unit synthesizes the oxygenate-containing hydrocarbon stream over a catalyst selected from the group consisting of an iron based catalyst or a cobalt based catalyst.

5. The process according to claim 1 wherein the reforming catalyst is an intermediate pore zeolite.

6. The process according to claim 5 wherein the zeolite is an intermediate pore zeolite is a crystalline silicate having a silica to alumina mole ratio of about 200 or greater; and an alkali content of less than 6000 ppm in the crystalline silicate; and an alkali to aluminum ratio in the crystalline silicate between 1 and 5 on a molar basis.

7. The process according to claim 5 wherein the catalyst is selected from the group consisting of ZSM-5, ZSM-11, ZSM-21, ZSM-22, ZSM-23, ZSM-25, ZSM35, ZSM-38, SSZ-20, SS-23 and combinations thereof.

8. The process according the claim 6 wherein the catalyst is selected from the group consisting of ZSM-5, ZSM-11, SSZ-20, and SSZ-23.

9. The process according to claim 6 wherein the catalyst includes a group 8 noble metal.

10. The process according to claim 8 wherein the platinum.

11. The process according to claim 1 wherein step d has a pressure from 0 psig to 200 psig, the liquid hourly space velocity is from about 0.1 to about 20 hr⁻¹, and the temperature is from 800° F. to 1100° F.

12. The process according to claim 10 wherein the pressure is from 25 psig to 75 psig, the LHSV is from 0.3 to 5 m⁻¹, and the temperature is from 840° F. to 1050° F.

13. The process according to claim 11 wherein the hydrogen recycles to fresh hydrocarbon feed is from 0 to 10.

14. The process according to claim 9 wherein the pressure is from 0 psig to 200 psig, the liquid hourly space velocity is from about 0.1 to about 20 hr⁻¹, and the temperature is from 600° F. to 1100° F.

15. The process according to claim 13 wherein the pressure is from 25 psig to 75 psig, the LHSV is from 0.3 to 5 hr⁻¹, and the temperature is from 840° F. to 1050° F.

16. The process according to claim 14 wherein the hydrogen recycles to fresh hydrocarbon feed is from 0 to 10.
